(19) United States
(12) Patent Application Publication
(10) Pub. No.: US 2013/0018276 A1
(43) Pub. Date: Jan. 17, 2013

(54) Tools and methods for the surgical placement of intraocular implants

(71) Applicants: Roberto Zaldivar, Emilio Citit (AR); Roger Zaldivar, Emilio Citit (AR); Youssef S. Wakil, Houston, TX (US)

(72) Inventors: Roberto Zaldivar, Emilio Citit (AR); Roger Zaldivar, Emilio Citit (AR); Youssef S. Wakil, Houston, TX (US)

(21) Appl. No.: 13/317,418

(22) Filed: Oct. 17, 2011

Related U.S. Application Data
(60) Provisional application No. 61/465,891, filed on Mar. 25, 2011.
(60) Provisional application No. 61/455,218, filed on Oct. 15, 2010.

(51) Int. Cl. A61B 5/107

(52) U.S. Cl. 600/558

(57) ABSTRACT

Provided herein is a measurement tool for implantable non-spherical asymmetric optics comprising a viewable, rotatable angular caliper superimposable over an image of an eye. Also provided are methods for optimally placing non-spherical asymmetric optics in an eye of a patient and for correcting post-operative astigmatism in a patient having cataract surgery. The measurement tool is useful to plan the optimal correct surgical placement of a non-spherical asymmetric optic, e.g., a toric intraocular implant or a toric intraocular contact lens, in the eye. By superimposing the measurement tool over a corneal topographic image, an optimal positioning of the non-spherical asymmetric optic can be effected in an optical zone of interest. Correct placement or re-placement at least minimizes astigmatism in post-operative vision. Also provided are computer program products and computer readable media comprising modules and methods for data entry, lens selection and surgical planning utilized to practice the methods provided herein.

### CROSS-REFERENCE TO RELATED APPLICATIONS

This international application claims benefit of priority under 35 U.S.C. §119(e) of provisional application U.S. Ser. No. 61/465,891, filed Mar. 25, 2011, now abandoned, and provisional application U.S. Ser. No. 61/455,218, filed Oct. 15, 2010, now abandoned, the entirety of both of which are hereby incorporated by reference.

### BACKGROUND OF THE INVENTION

1. Field of the Invention

The present invention relates generally to the fields of ophthalmology and ophthalmic surgery. More specifically, the present invention relates to a measurement tool and methods for measuring and planning placement of toric ocular implants to at least minimize post-operative astigmatism.

2. Description of the Related Art

Modern cataract surgery has embraced the benefits of placing not only spherical or aspheric intraocular lenses (IOLs) into the eye, but also toric IOLs which help to control astigmatism in the eye. The goal of toric, or astigmatic, IOLs is to correct, approximately, either the complete cylinder optics in the eye usually coming from the cornea and to maximize contrast sensitivity or to provide a desired amount of cylinder that can provide for reasonable depth of field in the eye which gives the patient a reasonable amount of far to near vision. With this higher level of sophistication of IOL designs the precise location of the axis of the IOL and overall positioning within the eye and its relation to the cornea and/or pupil and/or other structures of the eye must be obtained to achieve the ideal outcome.

Correcting even small or moderate amounts of astigmatism, such as less than 2D, does require high precision in proper placement. With the advent of multi-focal IOLs, this is even more critical as many multi-focal designs, for example, diffractive IOLs, do not perform well with any residual astigmatism in the eye. Small errors on the level of 3-5 degrees in placing the axis of the IOL in the eye can lead to large 10-20% loss of effective correction of the toric IOL. The higher level optical performance of modern “Premium” IOLs″ require the ophthalmic surgeon to improve his/her surgical planning and techniques to obtain optimal vision performance for his patient with implantation of a toric IOL. This improved methodology also applies to other toric implants in the eye, such as Toric ICL's or anterior chamber lenses and even corneal inlays. Correcting astigmatism in the eye with an implant generally requires placing a toric optical surface at the correct degree of rotation to cancel other sources of astigmatism in the eye such that when the optical image focuses on the retinal there is no optical cylinder, or a desired amount, if such is planned. With a toric IOL placement to replace the natural lens of the eye and as with most cataract surgeries today, the rotational placement of the IOL within the eye at the precise meridian to cancel the astigmatism from the cornea is planned prior to placement for an ideal outcome.

However, correctly planning the placement of toric IOLs today must overcome a series of poorly controlled measurements and marks that are all error prone and subject to changes. This results in a poorly controlled outcome in positioning the toric IOL for optimal vision correction. Given the challenges in accurately marking, measuring and placing toric IOLs, most surgeons, therefore, do not attempt the extra work required to maintain the controlled measurements necessary to adequately provide for the ideal toric IOL positioning and for this the patient's ultimate vision is sacrificed.

It is a recognized goal in the art of toric IOL surgery generally to place the IOLs toric power at the correct location in the eye to minimize or reduce to zero the astigmatism generated by the cornea. Thus, having a more direct correlation of the positioning of the IOL to the corneal topography and its optical powers would be a preferred system. The prior art is deficient in the lack of methods for measuring accurately and planning the placement of toric intraocular implants such that astigmatism in a patient's post-operative vision is corrected or minimized. The present invention fulfills this longstanding need and desire in the art.

### SUMMARY OF THE INVENTION

The present invention is directed to a measurement tool for implantable non-spherical asymmetric optics. The measurement tool comprises a viewable rotatable angular caliper superimposable over an image of an eye. The caliper comprises a pair of axes through the circle forming the angular caliper and intersecting at a point corresponding to a corneal vertex when superimposed over the eye and a plurality of markings around the circumference each corresponding to angular degrees from the axes.

The present invention also is directed to a method for optimally placing non-spherical asymmetric optics in an eye of a patient. The method comprises making reference marks at one or more points of interest on an eye and measuring the corneal topography of the marked eye to map its metrics of a steep axis, a flat axis and an angle of corneal astigmatism. The measurement tool described herein is superimposed over the corneal topographic image of the eye and an optimal angle of an optical zone on the cornea is determined for placement of the non-spherical asymmetric optics. The non-spherical asymmetric optic is positioned to coincide with the optimal angle of the optical zone. The present invention is directed to a related method further comprising step of measuring residual total astigmatism of the eye after placing the asymmetric optic into the eye to determine whether to further minimize or eliminate the residual astigmatism or to leave it to provide depth of focus.

The present invention is directed further to a method for correcting astigmatism in vision of a patient having cataract surgery. The method comprises measuring a corneal topography to pre-determine astigmatism in a cornea of the patient's eye and determining an angle within an optical zone of interest on the cornea of the eye for an optimal astigmatic correction based on metrics determined from the corneal topography. Using the measurement tool described herein, the surgical placement into the eye of an implantable non-spherical asymmetric optic is planned and the implantable non-spherical asymmetric optic is positioned to coincide with the optimal angle for the optical zone of interest. The present invention is directed to another related method to further minimize or eliminate post-operative residual astigmatism. The residual astigmatism is measured after the implantation. A new rotation and axis for the implanted non-spherical asymmetric optic required to minimize or to eliminate the residual astigmatism is calculated. The implanted non-spherical asymmetric optic is repositioned thereby further minimizing the post-operative residual astigmatism.

The present invention is directed further still to a computer program product for use in execution in a computer of a method for planning a surgical implantation of non-spherical asymmetric optics into one or both eyes of a patient where the computer has at least a memory and a processor. The computer program product comprises a data module, a lens selection module and a surgical plan module. The data module is configured to input into user-entered fields first values for at least IOL spherical power, surgically induced astigmatism and incision location and to output into calculated fields second values, calculated from the first inputted values, for at least lens data, an axis of placement of the non-spherical asymmetric optics in the one or both eyes and an expected residual astigmatism. The lens selection module is configured to select the non-spherical asymmetric optics based on the calculated values. The surgical plan module is configured to plan and to display a surgical implantation of the non-spherical non-spherical asymmetric optics based on the calculated values and the lens selection. The present invention is directed to a related computer program product where the data entry module is configured further to edit the inputted first values and recalculate outputted second values based on a post-operative residual astigmatism value.

The present invention is directed further still to a computer readable medium that tangibly stores the instructions for execution in a computer of a method for planning a surgical implantation of non-spherical asymmetric optics into one or both eyes of a patient where the computer has at least a memory and a processor. The method comprises steps for inputting into user-entered fields first values for at least IOL spherical power, surgically induced astigmatism and incision location, outputting into calculated fields second values, calculated from the first inputted values, for at least lens data, an axis of placement of the non-spherical asymmetric optics in the one or both eyes and an expected residual astigmatism. The method comprises a step for selecting the non-spherical asymmetric optics based on the calculated values and a step planning and displaying a surgical implantation of the non-spherical asymmetric optics based on the calculated values and the lens selection. The present invention is directed to a related computer readable medium comprising one or more of the method steps inputting first values for one or more of axial length, anterior chamber depth, central corneal thickness, lens thickness, or retinal thickness, outputting calculated values for one or more of pre-operative corneal astigmatism, a cross cylinder result for a corneal plane, cylinder power at the IOL plane, or cylinder power at the corneal plane or editing the inputted first values and recalculating outputted second values based on a post-operative residual astigmatism value.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1C depict the first or original image of the corneal topographic image (FIG. 1A) where the horizontal axis is at 180 degrees, the second image where the axes are adjusted by the surgeon by clicking and dragging the computer mouse (FIG. 1B) and the surgeon's view of the final surgical plan (FIG. 1C) for Patient 1.

FIGS. 2A-2C depict the first or original image of the corneal topographic image (FIG. 2A) where the horizontal axis is at 180 degrees and the steep axis is at 98 degrees, the second image where the axes are adjusted by the surgeon (FIG. 2B) and the surgeon's view of the final surgical plan (FIG. 2C) for Patient 2.

FIGS. 3A-3C depict the first or original image of the corneal topographic image (FIG. 3A) where the horizontal axis is at 180 degrees, the second image where the axes are adjusted by the surgeon (FIG. 3B) and the surgeon's view of the final surgical plan (FIG. 3C) for Patient 3.

FIGS. 4A-4C depict the first or original image of the corneal topographic image (FIG. 4A) where the horizontal axis is at 180 degrees, the second image where the axes are adjusted by the surgeon (FIG. 4B) and the surgeon's view of the final surgical plan (FIG. 4C) for Patient 4.

FIGS. 5A-5B depict a dialog box for the Toric Calculator showing pre-operative data entry with the information displayed (FIG. 5A) and an initial Toric Planner Screen (FIG. 5B).

FIGS. 6A-6E depict various Toric Planner screens and dialog boxes displayed during a surgical planning procedure. FIG. 6A is a Toric Planner pre-adjusted screen.

FIG. 6B is a Select IOL dialog box presenting the three toric lens options in which Lens Option 2 is checked. FIG. 6C is a screen depicting the adjusted toric caliper. FIG. 6D is the Edit Incision dialog box depicting data input edited for the degree and amount of Surgically Induced Astigmatism (SIA) at the incision site. FIG. 6E is a screen and the final Toric Planner once the incision site is modified.

FIGS. 7A-7B are Toric Planner screens depicting a surgeon's view of the operation plan with (FIG. 7A) and without (FIG. 7B) the eye image displayed.

FIG. 8 depicts the surgeon's view of the final plan for post-operative correction of residual astigmatism.

### DETAILED DESCRIPTION OF THE INVENTION

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “or” in the claims refers to “and/or” unless explicitly indicated to refer to alternatives only or the alternatives are mutually exclusive, although the disclosure supports a definition that refers to only alternatives and “and/or”.

As used herein “another” or “other” may mean at least a second or more of the same or different claim element or components thereof. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “toric” refers to the shape of an intraocular lens having two different curves instead of one which is utilized to correct both astigmatism and near- or farsightedness. A “toric intraocular contact lens” (ICL) refers to a very thin toric lens that are placed behind the iris and on top of the natural lens of the eye.

As used herein, the term “patient” refers to an individual or subject who has surgically received an intraocular implant and/or has surgically had placement of an intraocular implant corrected post-operatively and/or has been evaluated as a candidate for intraocular implantation. Preferably surgical procedures are or have been performed utilizing the toric calculator and toric caliper presented herein.

In one embodiment of the present invention there is provided a measurement tool for implantable non-spherical asymmetric optics, comprising a viewable rotatable circular caliper superimposable over an image of an eye, where the caliper comprises a pair of axes through the circle forming the caliper and intersecting at a point corresponding to a corneal vertex when superimposed over the eye; and a plurality of markings around the circumference each corresponding to angular degrees from the axes. In this embodiment, the circumference of the caliper superimposes approximately around the limbus of the eye.

In another embodiment of the present invention, there is provided a method for optimally placing non-spherical asymmetric optics in an eye of a patient, comprising the steps of making reference marks at one or more points of interest on an eye; measuring the corneal topography of the marked eye to map its metrics of a steep axis, a flat axis and an angle of corneal astigmatism; superimposing the measurement tool described supra over the corneal topographic image of the eye; determining, via the measurement tool, an optimal angle of an optical zone on the cornea for placement of the non-spherical asymmetric optic; and positioning the non-spherical asymmetric optic to coincide with the optimal angle of the optical zone.

Further to this embodiment, the method comprises the step of measuring residual total astigmatism of the eye after placing the non-spherical asymmetric optic into the eye to determine whether to further minimize or eliminate the residual astigmatism or to leave it to provide depth of focus. In an aspect of this environment, the residual astigmatism is further minimized or eliminated and method comprises the steps of subtracting corneal astigmatism from the residual total astigmatism to determine the current angle of the implanted non-spherical asymmetric optic; calculating a rotation of the implanted non-spherical asymmetric optic required to minimize or eliminate the residual astigmatism; calculating the angle between the marks on the eye and a new axis of the implanted non-spherical asymmetric optic; and rotating the implanted non-spherical asymmetric optic the calculated amount to coincide with the new calculated angle.

In both embodiments and aspects the optical zone metrics may comprise determining the sphero-cylindrical shape that is best fit to the optical zone of the corneal topography or of a corneal wavefront. Further, the step of determining the optimal angle for placement of the non-spherical asymmetric optic may comprise measuring one or more angles formed by one or more first axes each having a vertex coincident with one of the reference marks and a second axis comprising one of the metrics, where the first and second axes each have a vertex coincident with a central vertex in the eye whereby the non-spherical asymmetric optic position coincides with the axes. For example, the other vertex(ices) of the first axis(es) may comprise one of the reference mark(s). Also, the second axis may be coincident with a steep axis of the corneal topography curvature. In addition, the central vertex may be located in the center of the cornea, the pupil or the entrance pupil or the center of corneal topographic map or is located at a corneal anomaly.

In both embodiments and aspects thereof, the corneal topography may include one or both of wavefront or aberrometry measurements or measurements of other optical aberrations. Also, the optical aberration may be astigmatism. In addition, the non-spherical asymmetric optics may be implantable toric intraocular lenses or implantable toric intraocular contact lenses.

In yet another embodiment of the present invention, there is provided a method for correcting astigmatism in vision of a patient having cataract surgery, comprising the steps of measuring a corneal topography to pre-determine astigmatism in a cornea of the patient's eye; determining an angle within an optical zone of interest on the cornea of the eye for an optimal astigmatic correction based on metrics determined from the corneal topography; planning, via the measurement tool of claim 1, surgical placement into the eye of an implantable non-spherical asymmetric optic; and positioning the implantable non-spherical asymmetric optic to coincide with the optimal angle for the optical zone of interest.

In a further embodiment, the method comprises the steps of measuring residual astigmatism after the post-operative implantation, calculating a new rotation and axis for the implanted non-spherical asymmetric optic required to minimize or to eliminate the residual astigmatism; and repositioning the implanted non-spherical asymmetric optic thereby further minimizing the post-operative residual astigmatism. In both embodiments the steps of determining the metrics of the optical zone of interest and the optimal angle for placement of the non-spherical asymmetric optic comprises are as described supra. Particularly, the central vertex, the first ax(es), the second axis the non-spherical asymmetric optics, reference marks and their positions on the cornea or the sclera are as described supra.

In yet another embodiment of the present invention there is provided a computer program product for use in execution in a computer of a method for planning a surgical implantation of non-spherical asymmetric optics into one or both eyes of a patient, where the computer has at least a memory and a processor, the computer program product comprising a data module configured to input into user-entered fields first values for at least IOL spherical power, surgically induced astigmatism and incision location and to output into calculated fields second values, calculated from the first inputted values, for at least lens data, an axis of placement of the non-spherical asymmetric optics in the one or both eyes and an expected residual astigmatism; a lens selection module configured to select the non-spherical asymmetric optics based on the calculated values; and a surgical plan module configured to plan and to display a surgical implantation of the non-spherical asymmetric optics based on the calculated values and the lens selection.

Further to this embodiment, the data entry module is configured to edit the inputted first values and recalculate outputted second values based on a post-operative residual astigmatism value. In both embodiments the inputted first values further may comprise one or more of axial length, anterior chamber depth, central corneal thickness, lens thickness, or retinal thickness. Also, in both embodiments the outputted calculated values further may comprise one or more of pre-operative corneal astigmatism, a cross cylinder result for a corneal plane, cylinder power at the IOL plane, or cylinder power at the corneal plane.

In yet another embodiment of the present invention, there is provided a computer readable medium tangibly storing the instructions for execution in a computer of a method for planning a surgical implantation of non-spherical asymmetric optics into one or both eyes of a patient, where the computer has at least a memory and a processor, the method comprising the steps of inputting into user-entered fields first values for at least IOL spherical power, surgically induced astigmatism and incision location; outputting into calculated fields second values, calculated from the first inputted values, for at least lens data, an axis of placement of the non-spherical asymmetric optics in the one or both eyes and an expected residual astigmatism; selecting the non-spherical asymmetric optics based on the calculated values; and planning and displaying a surgical implantation of the non-spherical asymmetric optics based on the calculated values and the lens selection.

Further to this embodiment the method stored on the computer readable medium comprises the step of inputting first values for one or more of axial length, anterior chamber depth, central corneal thickness, lens thickness, or retinal thickness. In another further embodiment the method stored on the computer readable medium comprises the step of outputting calculated values for one or more of pre-operative corneal astigmatism, a cross cylinder result for a corneal plane, cylinder power at the IOL plane, or cylinder power at the corneal plane. In yet another further embodiment the method stored on the computer readable medium comprises the step of editing the inputted first values and recalculating outputted second values based on a post-operative residual astigmatism value.

Provided herein are methods, systems and tools for measuring and planning placement of non-spherical asymmetric optics, for example, but not limited to, toric ocular implants (most commonly Intra-Ocular Lenses—IOLs or Intra-Ocular Contact Lenses—ICLs) in the correct axis of the patient's eye for the patient to obtain the desired correction of astigmatism for the patient's post-operative vision. The measurement tool provided herein provides a means to correlate reference marks on the eye to corneal topography or refraction/wavefront measurements, such as internal optical aberrations or other measured visual properties of the eye that may interest an ophthalmic surgeon, in planning a surgical procedure, either pre-operative or post-operative, and in providing a complete metric system to accurately place toric or other asymmetric optics into the eye. Particularly, the measurement of corneal topography, more specifically, the steep axis of the cornea's curvature can be directly correlated to the marks on the cornea/sclera so a surgeon can reliably measure the angular difference and use this direct correlation to more accurately position the toric IOL to the appropriate axis to obtain precisely the visual outcome desired.

Thus, also provided herein are software applications, modules, computer readable media, and computer program products, etc. that enable a surgeon to use the toric calculator and toric calipers to plan a pre-operative surgical implantation of a non-spherical asymmetric toric lens or a post-operative implant correction thereof, as described in Example 2. As is known in the art, such software, modules, etc. can be tangibly stored in a computer or other electronic media, such as in a computer memory or other media storage device, retrieved therefrom and implemented therein. As also is known in the art, a computer or other electronic media comprises a memory, a processor, and, optionally, at least one network connection.

### I. Standard Measuring and Placing Procedures

### Methods

In order to place the axis of the toric IOL in the proper meridian, the ophthalmic surgeon must generate the proper metrics and system to use on the eye or through an imaging device such as a surgical microscope to achieve such measurements to guide him during surgery in placing the IOL at the right meridian and with ideal centration and positioning to the pupil and cornea and the eye's other components. The traditional procedure used to create such a metric system begins with the surgeon or a technician making a mark on the eye to determine the horizontal or 180 degree meridian as the patient is prepared for surgery. This typically involves the patient seated in front of a standard slit lamp observational microscope at which the patient is fixating on a coaxial light source. The observer determines that the patient has proper fixation and then uses a marking tool, usually blotted with an ink dye, and effectively pushes the marking tool down onto the cornea and/or the sclera of the eye to provide a “horizontal mark” for instance. This mark can be a short line or dot at the periphery of the cornea, usually at or across the limbus onto the sclera or “white of the eye”, so it can easily be observed at both the 3 o'clock and 9 o'clock positions, i.e., the 0 and 180 degree semi-meridians.

### Procedural Errors in Standard Methods

In marking the positions on the cornea or sclera a significant error is introduced as the patient's eye can move easily or rotate during the marking procedure. The technician or surgeon performing the markings can introduce many sources of error or bias in their alignment technique, etc. Once these marks are on the eye, they will represent theoretically the 180 degree or correct horizontal axis reference for the surgery. Errors in assuming that these marks are correct will be perpetuated in the process to determine the correct axis of IOL position.

Generally a surgeon uses these marks as a reference by which to measure the axis for the IOL placement using some standard caliper tools that demarcate the number of degrees from horizontal desired. There are a number of well-known and standard surgical measurement tools and methodologies useful to measure each of the 360 degrees around the eye from a reference point so that a subsequent mark can be made on the eye, for example, at 85 degrees, which represents the desired axis of final rotational placement of the IOL in the eye. Any errors in determining this 85 degree axis adds to the problem of controlling astigmatism. A toric IOL may have a corresponding mark or line such that, upon placement in the capsular bag of the eye as a replacement for the human lens, the IOL is rotated to align the mark on the IOL with the mark on the corneal limbus and sclera which denotes the final positioning of the IOL.

In general, the target axis for rotational placement of the IOL is determined so that once the IOL is placed correctly along this axis it will correct the cylinder of the cornea. Most toric IOLs are designed so that there is a mark on the lens that indicates one of its principle axes, either its axis of lower power or higher optical power. Usually the axis of lower power is marked and the IOL is positioned so that axis of lower power mark and the mark on the cornea or sclera which is intended to represent the axis of cylinder power that is greatest from the cornea are coincident. The corneal axis is generally referred to as the “steep axis” of the cornea. The axis of steepest curvature of the cornea then will provide the greatest optical power in a toric cornea. Therefore, it is presumed that when the IOL is rotationally positioned so that the marks on the cornea or sclera are aligned with the correlating marks on the IOL then the toric IOL should ideally neutralize the corneal astigmatism as planned.

There are a number of critical steps in measuring this process and there are errors associated with each of these steps. Currently, there is poor correlation of the placement of the IOL to the corneal topography or toric shape and power of the cornea. The standard metric systems used today by surgeon's reference marks on the eye are assumed to be horizontal or vertical and there is no true confirmation of this assumption. With cyclotorsion of the human eye from positioning the patient in the vertical to horizontal position, as needed for surgery, there are even greater sources of error introduced and what is considered horizontal in the eye when the patient is seated is clearly not the horizontal position when the patient is supine in most patients.

In addition the purely subjective nature of the observer in applying their technique to mark the “horizontal” axis of the eye given the patients head position, the quality of the ink marks and their potential to spread or blot and even to be non-visible over the few minutes until surgery occurs can affect the process. This can occur easily as fluids, such as artificial tears and anesthetic drops, are used on the eye. Furthermore, the use of surgical measuring tools such as angular calipers that are marked in 5 or 10 degree increments also leave a great deal of error and subjectivity in their use as a surgeon tries to find a target axis within one degree of accuracy given the accuracy required to truly provide the best vision.

### II. Toric Caliper Measuring System

### General Overview

As an improvement over the current standard implant planning technologies, the imaging tool and measuring system provided herein incorporate corneal topography measurements, with or without wavefront and/or aberrometry measurements, using known analog and/or digital imaging techniques and ocular measurements to directly correlate and measure the corneal topography and, therefore, its optical powers, including astigmatism, to the reference marks or positions on a patient's eye. Previously, marks on the cornea/sclera were at the horizontal axis, however, the measurement tool and methods of use provided herein eliminate this requirement. The reference marks may be placed anywhere that is convenient for the surgeon and that can be seen in the corneal topography image. This direct correlative measurement provides for increased precision in planning the surgical procedure and provides a simple guide for the surgeon to appropriately and correctly place the toric IOL.

Through imaging techniques of measuring the corneal topography, for example, Placido Disk imaging that simultaneously images the marks on the cornea/sclera, image processing can be used either manually or automatically to detect these two axes and/or marks and to determine the angular distance necessary to place the toric IOL to ideally control the astigmatism in the eye. In a representative embodiment, an angular caliper is used to draw a first line through the corneal vertex or center of the corneal topography map and the desired reference mark on the cornea/scleral part of the eye, through either manual or automatic detection means. This first line is followed by a second line that includes the corneal vertex and is coincident with the steep axis of the corneal topography curvature. This second line may be considered a principle meridian of the cornea's average toricity; for example, actually defining the steep meridian of the cornea. In this simple case the angular difference between these two lines that share a common point at the corneal vertex correlates to an ideal placement of the IOL to control astigmatism, as planned. Any variation in this plan can be measured if, in fact, an alternative amount of cylinder is desired as the outcome.

Utilizing modern software graphic techniques and analysis the corneal topography measurement that incorporates the image size to detect the marks on the cornea/sclera is sufficient to begin the toric caliper analysis and leads to a direct plan for surgery. A color printout can be easily generated or the output can be sent as a digital image or video to a monitor system, either through the operating microscope or generated on a video screen by superimposing the toric caliper measurements onto a live video image using image processing techniques, to locate anatomical landmarks, such as, but not limited to, the pupil and limbus. Alternatively, more sophisticated iris registration techniques may be used. The computer hardware, monitor and video equipment necessary to produce an image are well-known and standard in the art.

The goal of achieving a single data capture incorporating corneal topography analysis, optionally, with wavefront/aberrometry analysis, and the direct imaging of the reference marks made on the cornea/sclera enables direct correlative measurements to direct surgical planning. In practically all cases the handmade markings on the cornea/sclera are not perfectly symmetrical over the cornea's center or that of the corneal vertex or pupil or other central ocular landmark. This, however, is not of consequence as surgical planning can proceed from a minimum of one marking or multiple markings and each can provide a direct correlative measurement to the corneal topography, whether the steep axis of cylinder is desired or the flat axis or any semi-meridional analysis. The surgeon can select any feature of the corneal topography to use as his guide for placement of the toric or any customized optic as he desires the visual outcome to be.

### Steps in Performing a Toric Caliper Surgical Plan:

1) A patient that has been predetermined (due most likely to a significant degree of corneal astigmatism) to receive a Toric IOL is first marked on the eye by a technician or doctor, such as, but not limited to, the 3 and 9 o,clock positions, to serve as a reference mark for the doctor in surgery.

2) The corneal topography measurement is taken with video imaging to see the marks on cornea, limbus or sclera. Optionally, this can be combined with aberrometry measurements or with other diagnostic measurements, such as axial length corneal pachymetry. This can also be performed with patient seated, or supine or in any position. In a supine position the device can be held manually or by a vertical stand.

3) With the CT and video image, for example, but not limited to, a digital image, the surgeon or technician can be shown a display with the CT map overlaying the video image. This could be a transparent map or semi-transpaterent or also a solid map, usually in color.

The color may denote the curvature of the cornea therefore its optical power, but can also denote elevation, etc.

4) The user can then select the Toric Caliper graphics and software to activate at anytime to now have an angular graphic display with angular calipers that can be set either manually or automated through software image processing and mathematical algorithms to most likely correlate the “surgeon's mark” (3 o'clock and 9 o'clock in this instance) to the steep axis (meridian of most refractive power) of the cornea as is typically done. The user can now use the angular information of the caliper to determine how many degrees from their surgeon's marks they need to use to place the Toric IOL in the proper alignment with the cornea.

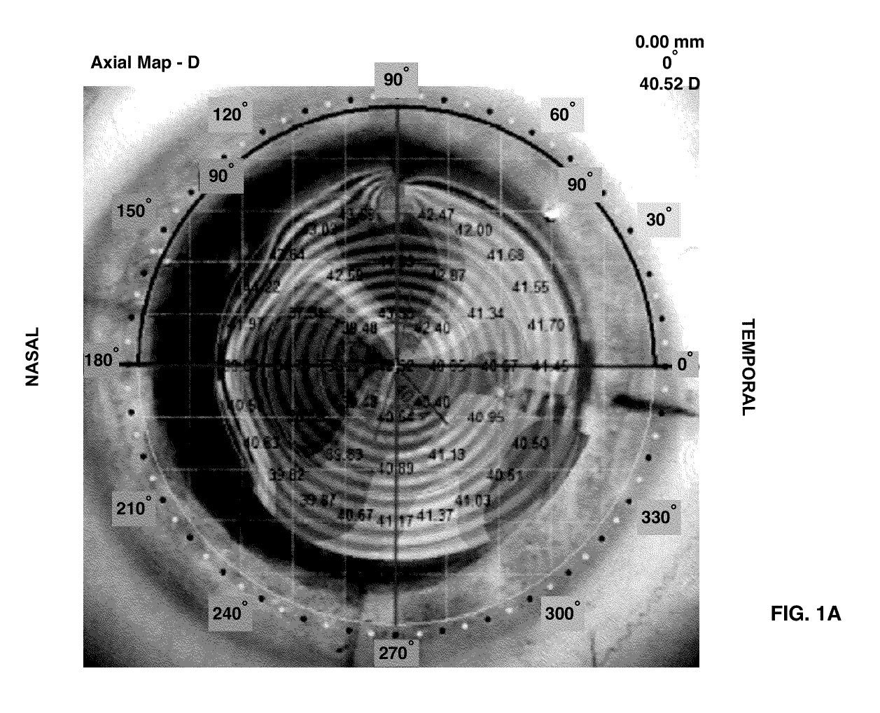

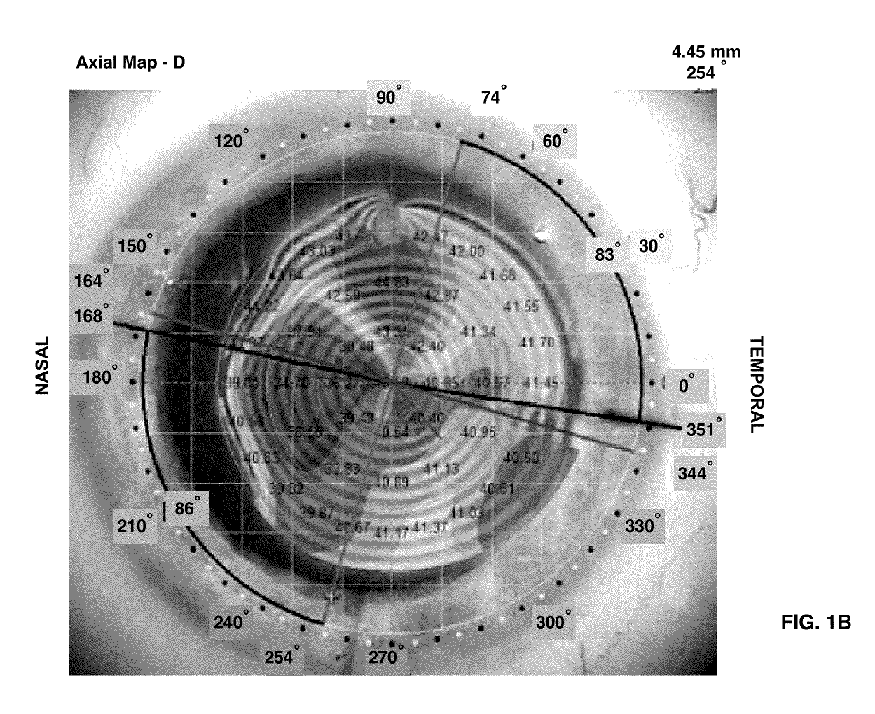

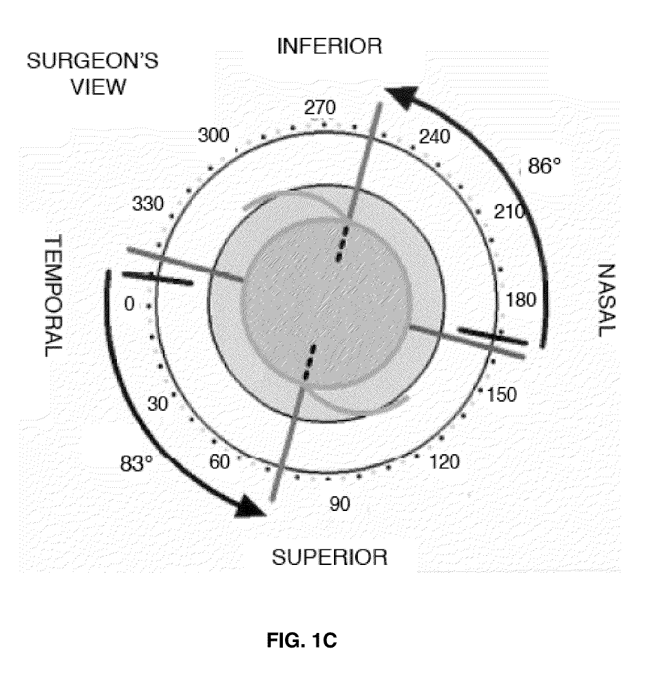

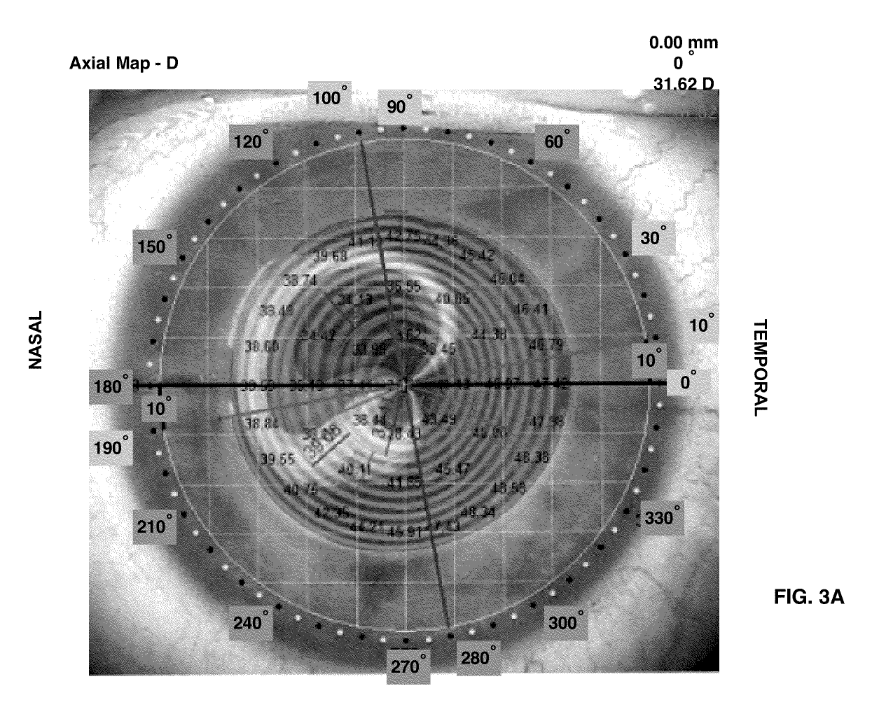

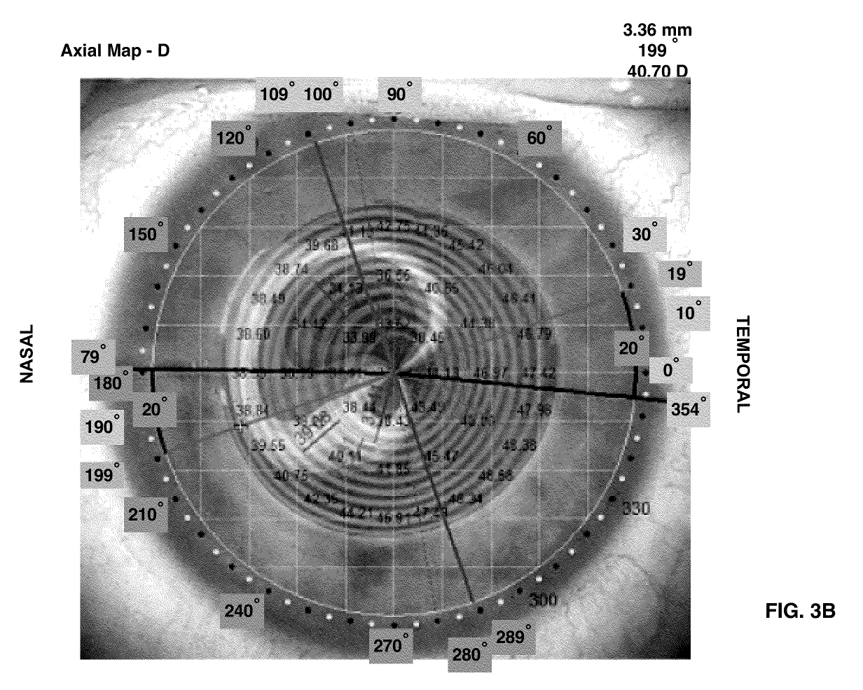

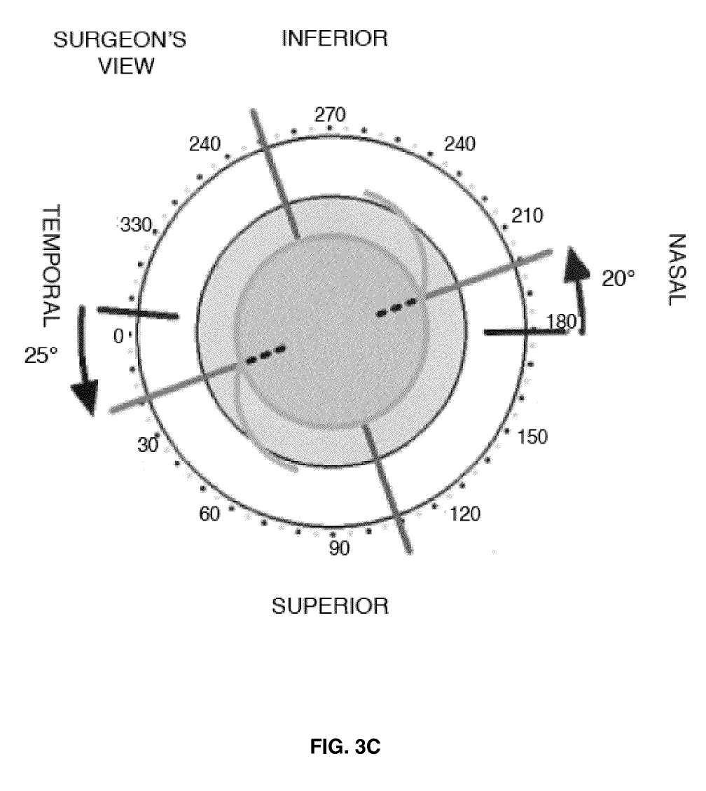

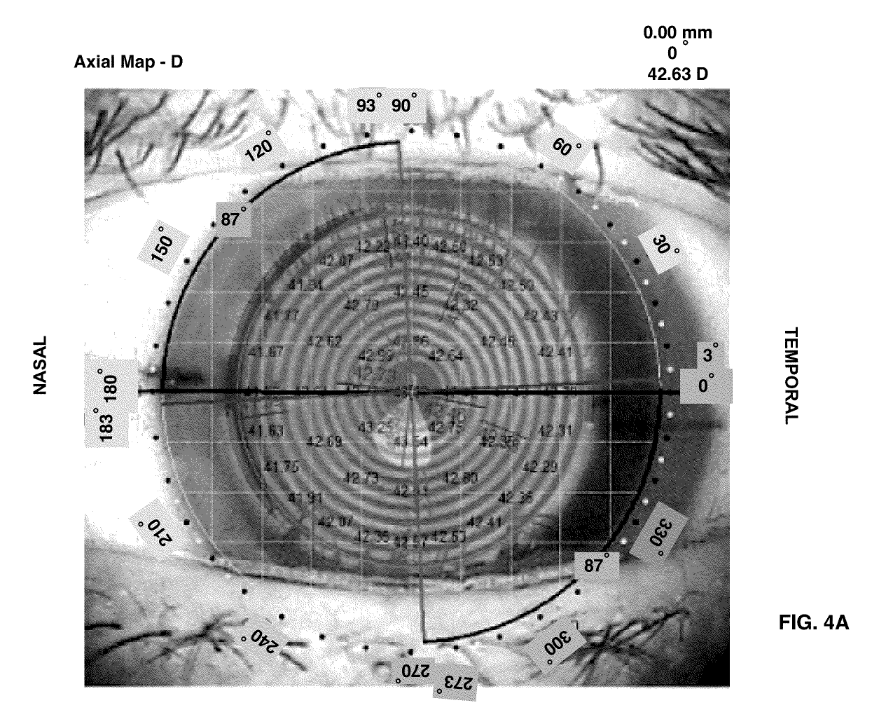

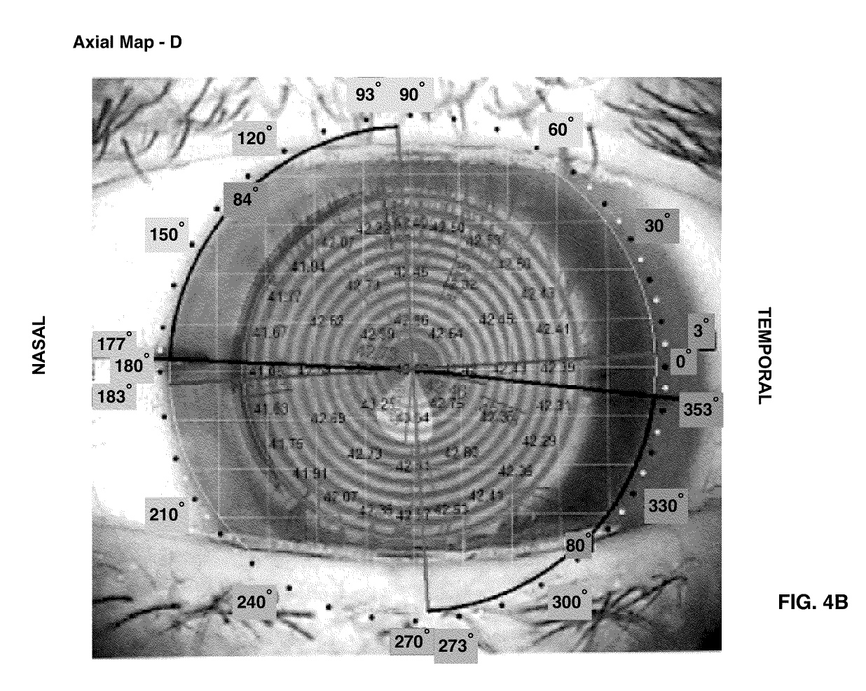

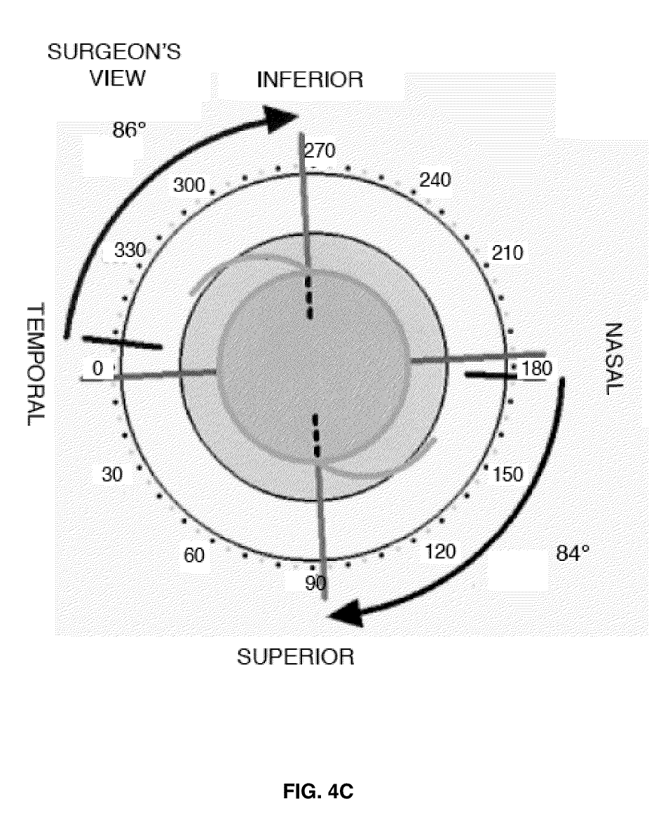

For example, manually the user can place a semi-meridian marker axis over the steep axis of corneal topography (FIGS. 1A, 2A, 3A, 4A), representing that this is eventually the axis where he wants the lower power principle meridian of the Toric IOL to be. Then he can take the “horizontal reference line” that is initially portrayed and move it over one, or both, or however many, surgeon's marks that have been made on the eye so that once placed the Toric Caliper will give him the angular distance from that mark to the desired final position of the lower power axis of the Toric IOL (FIGS. 1B, 2B, 3B, 4B). In this case that will be the same axis as the steep axis of corneal cylinder.

This Toric Caliper can be centered on the Vertex Normal of the cornea which is the center of most corneal topography maps, but the Caliper could also be centered on other desired points on the eye as the user desired. Some examples are the “Visual Axis” or first light reflex off the cornea when a patient is properly fixating. It could also be the center of the pupil or entrance pupil as determined or it could be other points of interest such as the apex of the cornea or some corneal anomaly like a scar.

Again, the user can override an automatic system or manipulate a manual one to make any adjustments he sees necessary; for example, with irregular astigmatism. Or if there is little or no corneal astigmatism and the surgeon is planning to induce some desired astigmatism in the eye, which could be highly beneficial in giving the patient more depth of field optically, so that they can overcome Presbyopia and see near and far in a normal like manner.

5) Finally, once the user has positioned the cursors, or it has been done automatically by the software, then the user can confirm it is correct and is desirable, the user can actually select a display algorithm to present this information in a format for surgery, a surgical plan (FIGS. 1C, 2C, 3C, 4C). In these figures, the Surgeon's view is portrayed as upside down as the surgeon likely sits at top of a supine patient's head when doing surgery. The display algorithm gives the surgeon easy to follow graphics which are used intra-operatively and which indicate what is the correct angular distance, or any metric desired, for him to place a mark on the eye reference from the earlier pre-op Surgeon's mark. This latter mark represents the final mark that is used to align the IOL, or other ocular implant, when manipulating it in the eye so that it is positioned ideally for the desired astigmatism outcome. Usually in this procedure there is another final mark on the eye made in similar manner at the location 180 degrees from the first using either the same original pre-op “surgeon's mark” or its other paired “Surgeon's mark” so that the surgeon undergoes a duplicate step as above in that he has now to final “Alignment Marks” on the eye's cornea, limbus or sclera for him to use in positioning the Toric IOL; for instance, at the right angular position and even at the correct translational position in the eye.

### Post-Operative Correction

The toric caliper also is utilized for post-operative correction, if necessary. For example, if after a toric IOL or ICL implantation procedure, the axis is incorrect post-operatively, i.e., residual astigmatism is still present, the surgeon can utilize the toric calculator and toric caliper to determine the number of degrees and in what direction the toric implant must be rotated to further minimize the astigmatism. Preferably, this procedure is performed within 48 hours after surgery. Alternatively, it may be decided to leave the residual astigmatism to provide for depth of focus.

### III. Software

The software enables the toric caliper tool and creates the displays within a toric planner and IOL selection or evaluator modules. This enables a user to enter pertinent data from other sources to calculate the proper axis of alignment and cylinder power for the toric IOL or ICL implantation. The user enters a location that is 0 to 360 degrees from where the surgeon wants the cataract incision for surgery. However, whenever a cataract incision or any other type of incision to control astigmatism, such as a limbal relaxing incision (LRI) or incisions during astigmatic keratotomy (AK), is placed in the eye or cornea, a surgically induced astigmatism will occur. The toric planner module enables a user to incorporate such surgically induced astigmatism into the surgical plan for implantation.

In a representative example, a doctor makes the incision along the temporal side of the left eye at 5 degrees, slightly off the horizontal. Along that 5 degree meridian the cornea will flatten where the extent of flattening depends on size and length of the incision. With a standard cataract incision of 3 mm, flattening along the meridian across the incision averages 0.5 D. There also is a slight steepening in the perpendicular meridian at 95 degrees in this instance. This is referred as a coupling effect and may result in a total contribution of about 0.75 D of surgically induced astigmatism to the cornea. The software modules as described in Example 2 enable a user to account for such effects.

It is important to account for surgically induced astigmatism when planning the axis of a toric IOL implant. Surgically induced astigmatism creates a vector force which can now be predicted and summed with the pre-existing corneal astigmatism, if any, together with the optical cylinder in the toric IOL itself. What is now possible is to even select the toric IOL that is best suited for the eye and then use the toric caliper to mimic the cylinder of the cornea, IOL and surgically induced cylinder or astigmatism. This enables the surgeon to plan the surgery and to predict the outcome and, therefore, to better control the results. This can work not only for cataract surgery, but other forms like astigmatic keratotomy, even corneal transplant or corneal refractive surgery, especially with incisions.

Moreover, instead of relying on K readings, that is, the flat and steep axis of the cornea, to eventually align the IOL or ICL axis with respect thereto, there is improvement by looking at the “best fit” sphero-cylindrical shape or “optical” fit to the area over the cornea over a particularly desired optical zone. The optical zone can be chosen based on the patients pupil size, usually, the largest scotopic pupil size during darkness or may be selected by the optic zone of the IOL or ICL, if that is smaller, so that optical effects are optimized. This best fit can come from the cylinder terms of the Zernike Polynomial (Zernike Cylinder) fit which are incorporated into the toric planning software modules. This is an improvement over K readings obtained in keratometry. The best fit is generally more reproducible and takes into account the entire area of the cornea, such as, for example, over about a 5 mm zone, if the pupil size is that large, or over about a 3 mm zone for a smaller pupil. Alternatively, a least squares best fit method for a toric surface can be used. Mathematically, as is known in the art, there are several ways of doing this. This improves results optically in matching the toric IOL to the cornea over simple K readings. The steps to determine a best fit utilizing, for example, Zernike Cylinder terms, is enabled by the software modules in the Toric Planner

The following examples are given for the purpose of illustrating various embodiments of the invention and are not meant to limit the present invention in any fashion.

### Example 1

### Corneal Tomography with Toric Caliper

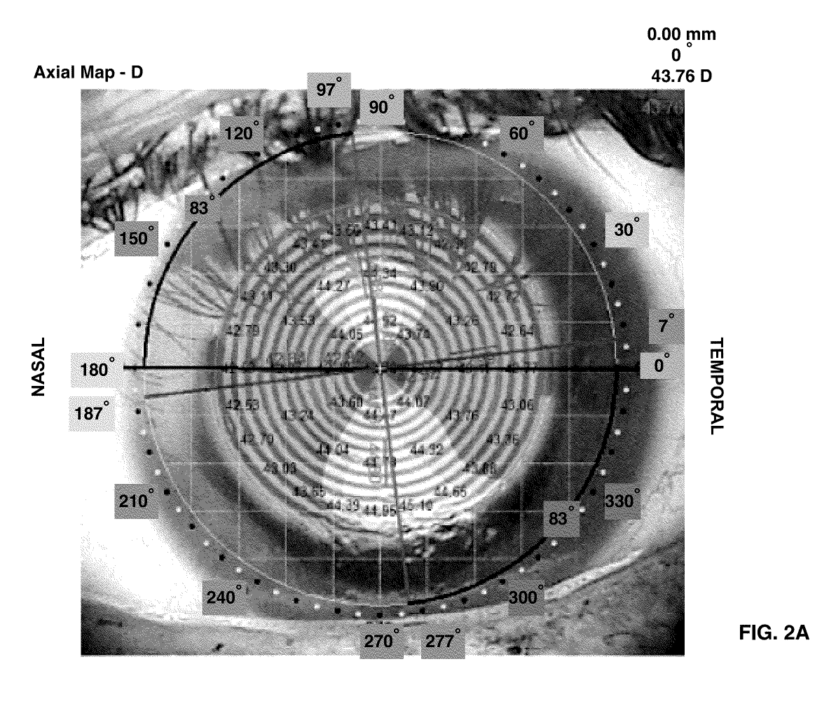

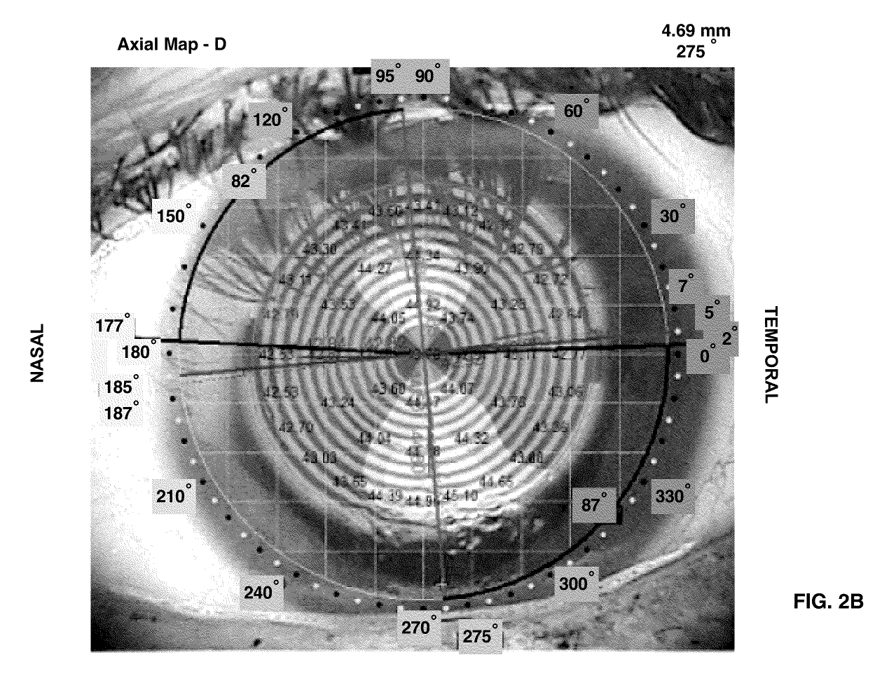

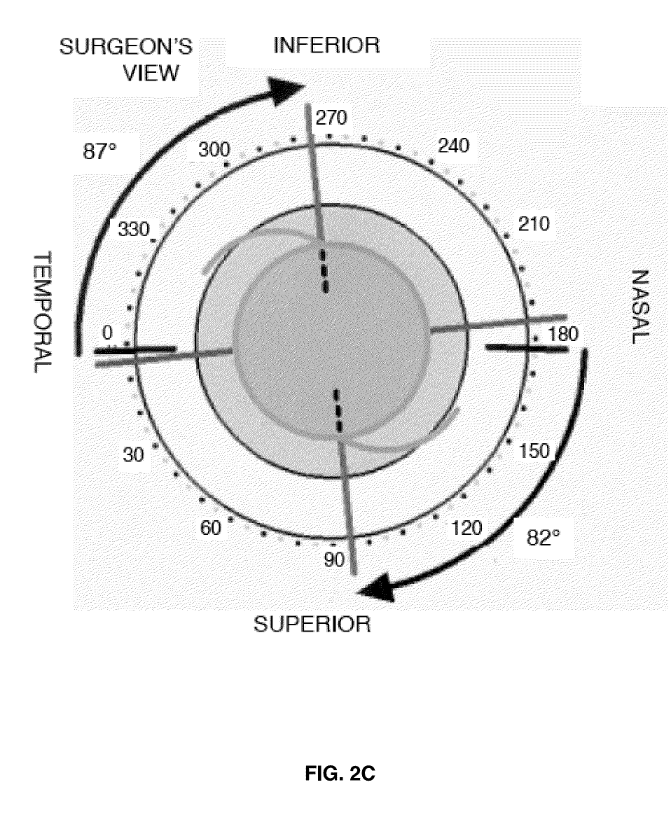

FIG. 1A shows the corneal topography (CT) over the eye image with a vertical red line and a horizontal black line. The horizontal line is the caliper tool at set up (0 degrees). This patient has vertical astigmatism where the red line is on 90 degrees, however, generally, the line usually is not at a perfect 90 degrees. For example, FIG. 2A shows Patient 2's eye with astigmatism where the steep axis is at 97 degrees. This is more typical, and note that the second patient's flat axis is 90 degrees away at 7 degrees. These red and blue lines are automatically generated by the corneal topography software as the flat and steep axes of the cornea as determined by keratometry which all CT systems emulate.

In this software, if the doctor does not agree or if the astigmatism is not as perfect and symmetric as it is in the case of Patient 2 then the doctor can alter these red and blue determinant lines of the steep and flat axes of the cornea. In that case a dotted version of the red and blue lines is left underneath so that the doctor can always see what the automated keratometry analysis shows. Also, he can use the mouse cursor and “pick up” the lines and rotate them to where he wants as this represents the corneal astigmatism which he then wants to correct or alter with the toric IOL that will go inside the eye.

As the Red and Blue Lines mostly are not touched and are determined, as per the automatic keratometer software, on the corneal topography, the user (in Manual Model uses the mouse cursor to “pick up” the black line off to the right of center and moves a semi-meridian black cursor line to usually cover half the red (steep axis) thereby demonstrating his “target” axis. Manually placing a black cursor line over the red axis tells the software this is where the user eventually wants the lower power axis (flat) of the toric IOL to reside.

The horizontal black axis or “reference axis” will remain completely across the screen and the user will then use the mouse cursor to go to the periphery (over the white of the eye) and “pick up” this full meridian reference axis and place it over the closest surgeon's mark that was made by an ink marker on the cornea or limbus or white of the eye (sclera). As in the case of Patient 1, the black “Hash mark”, which the surgeon made as his “Surgeon's Mark”, is below the horizontal by 9 degrees, so when he positions the reference line of the caliper down 9 degrees over the surgeon's mark, he is left with a completed plan for surgery.

The plan indicates that the angle from the Surgeon's mark (full black reference line) to the red axis of astigmatism that now has its upper half covered by the black semi-meridian “Target” cursor, denoting this is where he wants the final Toric IOL to be positioned to correct the steep meridian of the cornea. In the upper right are angle numbers that are colored to describe the angles now shown. Thus, for 99 degrees, the top number represents the angle from the now correctly placed reference line that is over the surgeon's mark to the Target Cursor, which is over the steep axis of the astigmatism, telling him that during surgery he needs to make a mark that is 99 degrees superior from the temporal (since it is the left eye that you see when the 3 o'clock Surgeon's mark is the temporal side of the eye) surgeon's mark.

Also, the surgeon will essentially do the same as above with the nasal surgeon's mark, putting the reference line over it and then taking the target semi- meridian line and overlaying it on the other half of the red steep axis of corneal astigmatism to get the angle that he should then measure and mark in surgery to make an inferior mark on the eye so he can line up the other side of the IOL. In this case that angle would be 112 degrees. Then, the doctor presses a button that says print surgical plan whereupon he receives a very simple summary of these two angles (99 degrees superiorly from the temporal markyand 112 degrees inferiorly from the nasal mark so that he takes this simple diagram that is usually in an upside down view (surgeon's view) to the OR.

### Example 2

### Software

### Action/Response Steps

### General Functional Requirements

The Data Entry module displays the entry fields and labels for the user-entered pre-op data and the calculated fields as shown in Table 1 (FIG. 1).

TABLE 1

| | |
|---|---|
| Required user entered fields: | IOL spherical power (D) |
| | surgically induced astigmatism (d) |
| | incision location (0-360°) |
| Optional user entered fields: | axial length |
| | anterior chamber depth |
| | central corneal thickness |
| | lens thickness |
| | retinal thickness |
| Calculated fields: | pre-op corneal astigmatism |
| | (x.xxd @ yy°) |
| | cross cylinder result (corneal plane) |
| | (x.xxd @ yy°) |
| | axis of placement (°) |
| | lens data for recommended iols |
| | #1, #2 or #3: |
| | expected residual astigmatism |
| | (x.xxd @ yy°) |
| | cylinder power at iol plane (d) |
| | cylinder power at corneal plane (d) |

The software also houses a database of lenses with varying cylinder power. A representative example is shown in Table 2.

TABLE 2

| | |
|---|---|
| at IOL plane | at corneal plane |

Cylinder Power

| | |
|---|---|
| 1.50 | 1.03 |
| 2.25 | 1.55 |
| 3.00 | 2.06 |

Potential Future Powers

| | |
|---|---|
| 3.75 | 2.57 |
| 4.50 | 3.09 |
| 5.25 | 3.60 |
| 6.00 | 4.11 |

### Data Entry

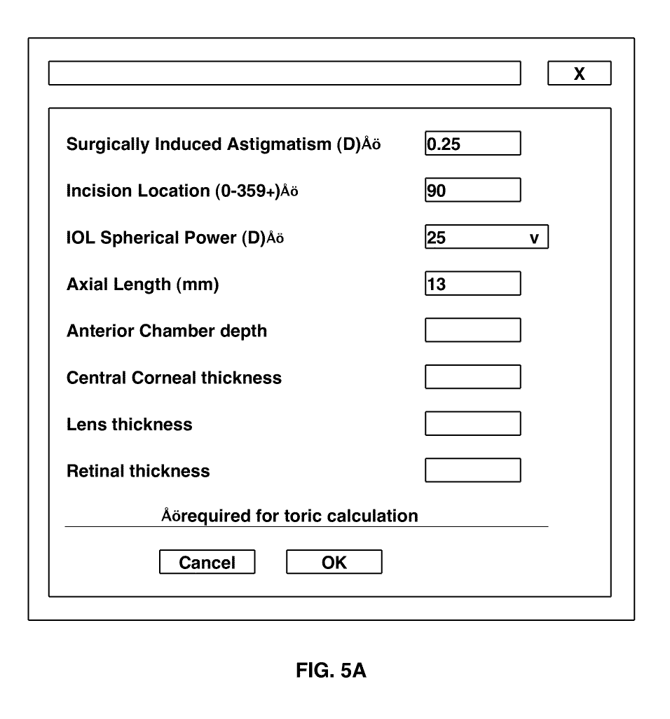

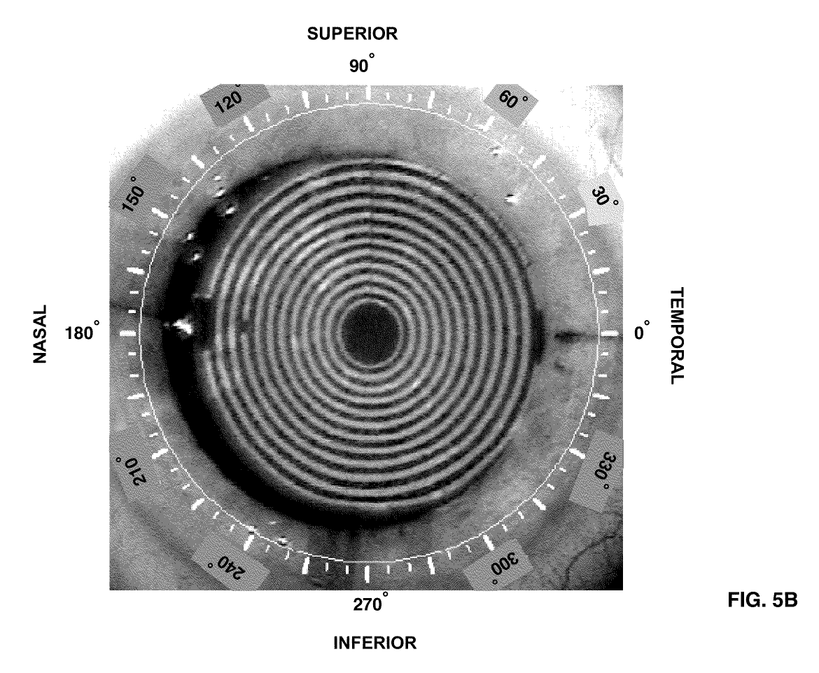

A Data Entry module enables a dialog box (FIG. 5A) in a Toric Planner Screen (FIG. 5B) that is accessible from the display and enables entry, at the Enter PreOp Data window, of the information not available through the wavefront (WF) and/or corneal topography (CT) exam data (for example, shown on an Exam Display Screen), as shown in Table 3.

TABLE 3

| Action | Software Response |
|---|---|
| 310 | 312 |
| From within the | Software opens dialog box with entry fields for: |
| display, the user clicks | Surgically induced astigmatism* (SIA) |
| the data entry | Incision location* |
| button. | IOL Spherical Power* |
| | Axial length |
| | Anterior Chamber depth |
| | Central Corneal thickness |
| | Lens thickness |
| | Retinal thickness |
| | *required for calculator to produce axis of |
| | placement |
| 320 | 322 |
| User enters a SIA in | The software displays the user-entered SIA in the |
| the form field labeled | formula and uses it in the calculation. |
| “Surgically Induced | 324 |
| Astigmatism (D)” in a | If the entered SIA is out of the range of 0.00 to |
| range from 0.00 to | 2.00, a warning message appears: “Surgically |
| 2.00 D, limited to | induced astigmatism value is out of range. |
| 1/100^{th} D steps | Please enter a value between 0.00 and 2.00 |
| (two decimal places.) | diopters.” |
| 330 | 332 |
| User enters the IL | The Incision Location (IL) value appears in the |
| angular location in | form field. The software uses this location to |
| the form field labeled | place the incision symbol on the surgical plan. |
| “Incision Location | 334 |
| (0-359°)”. | If the IL value is outside the range of 0-359, a |
| | warning message appears: “Incision location |
| | value is out of range. Please enter a value |
| | between 0° and 359°.” |
| 340 | 342 |
| User selects from a | The software displays the user-selected IOL |
| drop down list of | spherical power. User cannot enter a power |
| IOL Spherical Power | value, they must select it. |
| values that appear | |
| in 0.50 D steps from | |
| 15.00 D to 26.00 D in | |
| the form labeled | |
| “IOL Spherical | |
| Power (D)”. | |
| 350 | 352 |
| User enters the | The software displays the user-entered biometry |
| optional biometry | values. |
| values in the form | |
| field labeled | |
| accordingly. | |
| 320 | 322 |
| User clicks OK | Software returns to the display with the entered |
| within dialog box | data appearing on screen. The software places |
| | the toric indicator red line at the steep axis of the |
| | cross cylinder result (the axis of placement). The |
| | steep and flat sim K lines (the dashed lines) will |
| | remain at the sim K axes |
| | The Caliper tool will function as described herein. |
| User clicks Cancel | 332 |
| within dialog box | SW returns to display with no data entered. |
| | The caliper tool will function as described. |

### Selecting a Lens Option

Within the dialog for the user to choose the desired lens, the Lens Selection module displays 3 lens choices, or will display only two choices if the recommended lens is either a non-toric or the highest toric power. The software determines which lens choices to display based on the criteria in Table 4, as a representative example.

TABLE 4

| Lens Option | Correction of Astigmastism | Residual Astigmatism |
|---|---|---|
| 1 | optimum correction | Lowest positive value |
| 2 | under correction | 2^{nd} lowest positive value |
| 3 | over correction | negative value closest to zero |

For Lens Option 2, this entire field will be left blank if the patient has low pre-existing astigmatism and non toric lens (0 cyl power) is the optimal, i.e., Lens Option 1, selection. Also, for Lens Option 3, this entire field will be left blank, if the patient has high pre-existing astigmatism and the highest cyl lens is the optimal, i.e., Lens Option 1, selection.

The Lens Selection module enables the user to select one of the 3 lens options as shown in Table 5. On the display, this button is greyed out until the pre-op data entry requirement is fulfilled by the user.

TABLE 5

| Action | Software Response |
|---|---|
| 510a | 512 |
| From within the display, | Software opens dialog box with 2 to 3 lens |
| user clicks lens | choices with selection boxes. |
| selection button. | |
| 510b | 514 |
| User checks lens option | The software fills in the box with a check and |
| #1 | displays the chosen lens on the Display page. |
| 520 | 522 |
| User checks lens option | The software fills in the box with a check and |
| #2 | displays the chosen lens on the Display page. |
| 530 | 532 |
| User checks lens option | If the over correction of astigmatism TIOL |
| #3 | option is selected, a warning will appear that |
| | says, “Your selected lens has the potential to |
| | rotate the patient's axis of astigmatism 90°. |
| | Do you want to proceed? Yes? No?” |
| | 524a |
| | The user must select “yes” to proceed. The |
| | warning message closes and the software |
| | returns to the lens selection dialog. The |
| | software fills in the box with a check and |
| | displays the chosen lens on the Display page. |
| | 524b |
| | If no, warning message closes and the |
| | software returns to the lens selection dialog. |
| | 526 |
| | The check mark will not display next to |
| | Option 3. |
| 540 | 542 |
| User clicks OK. | The software returns to the display with lens |
| | selection displayed. |
| 550 | 552 |
| User clicks Cancel. | The software returns to the display with not |
| | lens selection appearing. |

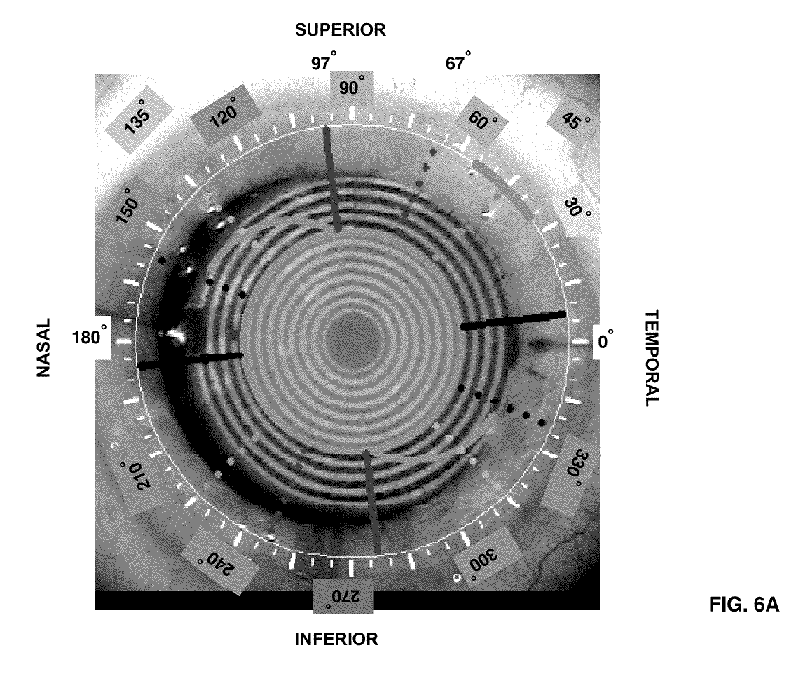

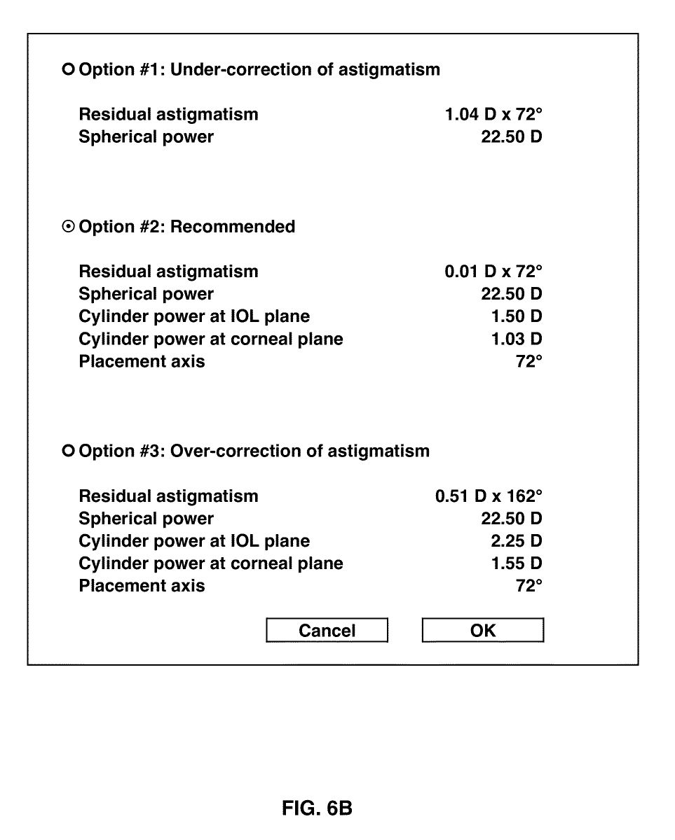

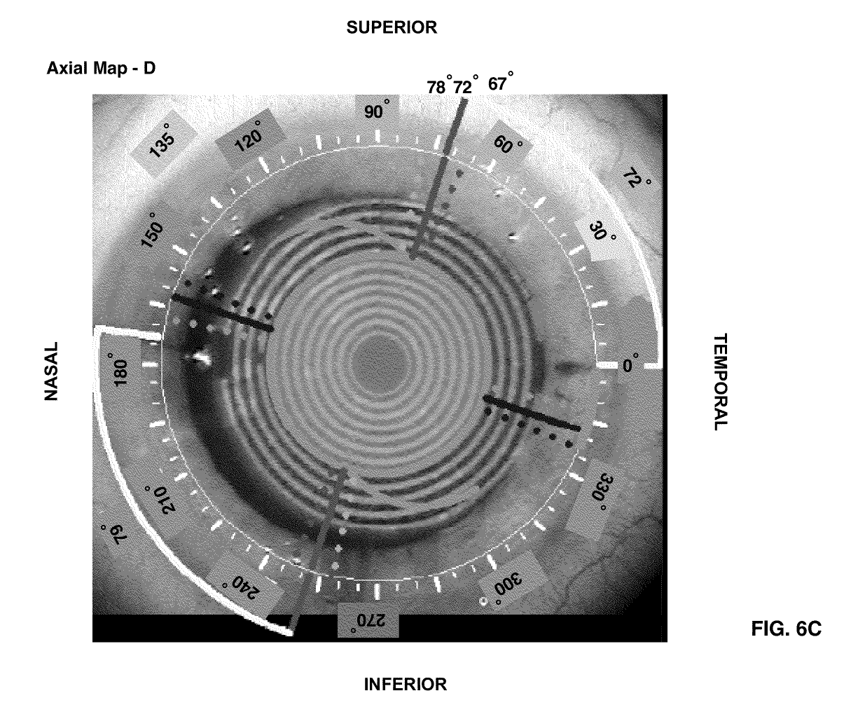

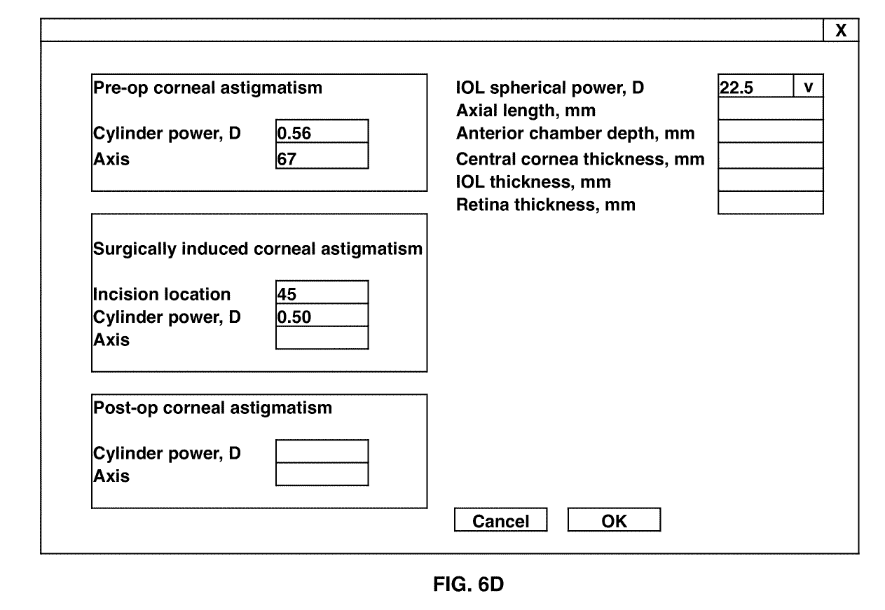

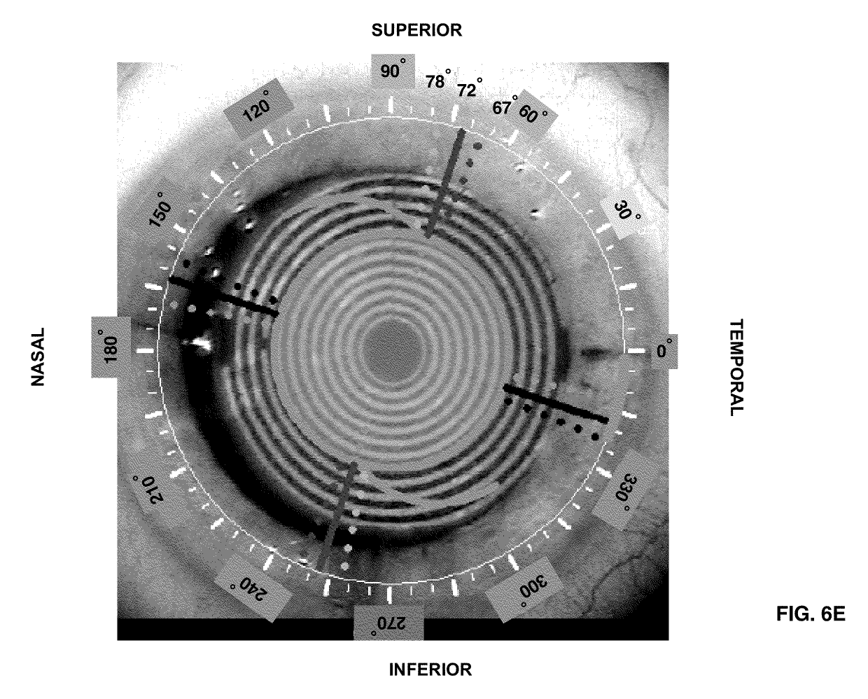

Once selected, the lens power at the corneal and IOL plane and the resulting expected residual astigmatism are displayed by the Surgical Plan module on the Surgical Plan page. The same dialog includes a drop down list with available lens models to choose from. An example of a lens selection screen where Lens Option 2 is recommended based on data input and selected by the user is shown in FIG. 6B. Moreover, a surgical plan may be edited by changing the data in the dialog box (FIG. 6D).

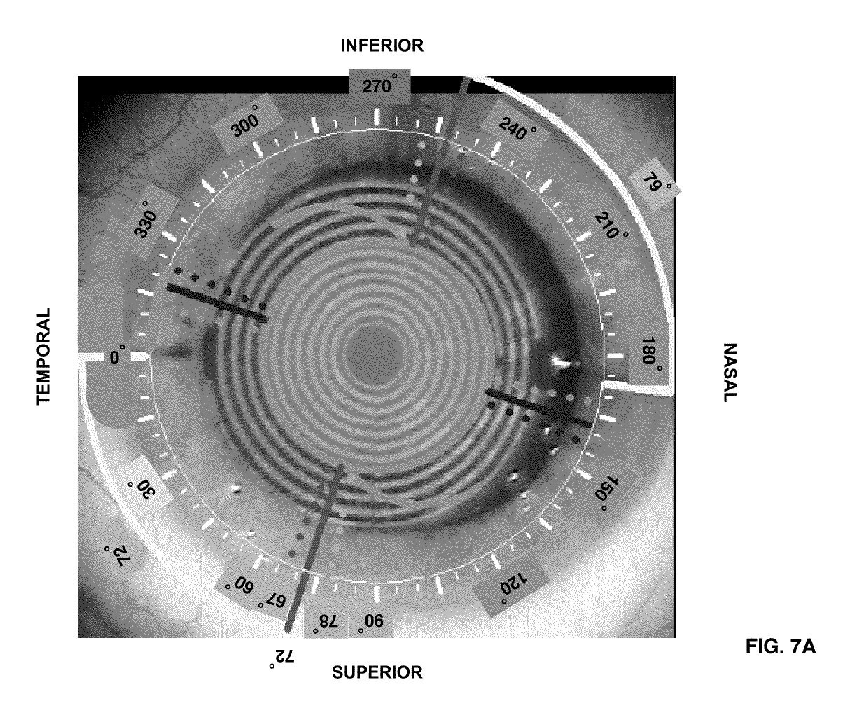

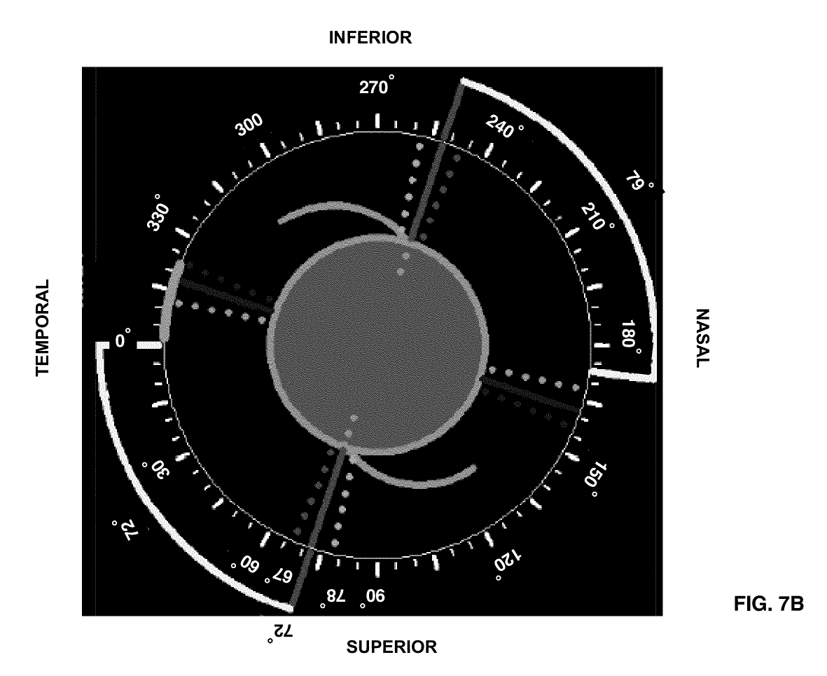

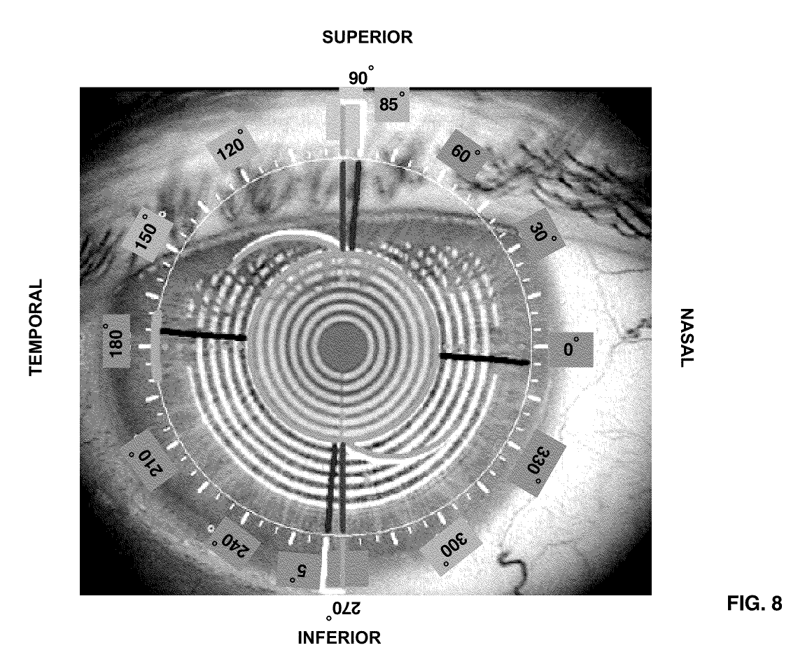

The Toric Planner shows a screen with a map displaying a pre-adjusted caliper (FIG. 6A). After initial data input and lens selection, the Toric Planner displays the adjusted caliper (FIG. 6C). If the incision site is modified, the Toric Planner displays a map depicting the modifications to the site on the eye (FIG. 6E). When the surgeon is satisfied with the surgical plane, a map, oriented for the surgeon's viewing, displays the operation plan (FIG. 7A) and may be printed out. FIG. 7B is a view of the operation plan in which the eye image has been removed.

### Post-Operative Plan for IOL Implant

The software modules described herein enables a user to design a post-operative plan, if the patient's astigmatism still requires correction after implantation of a toric IOL, as shown in Table 6. FIG. 1 shows an example of a final screen after adjustment by the surgeon.

TABLE 6

| Action | Software Response |
|---|---|
| 610a | 612 |
| Within the Enter PreOp Data | The software redraws the pre-op |
| window in the Toric Planner | data screen per GUI |
| display, the user clicks Post-Op | requirements. The new entry |
| Evaluation | fields include the lens sphere and |
| | cyl powers, lens manufacturer and |
| | model number. |
| | 614 |
| | The Current Lens Axis will |
| | populate with the Zernike |
| | combined Astigmatism axis at 4 mm |
| | (scan size if smaller) for the |
| | Internal Optics aberrations. The |
| | screen notes where this |
| | information comes from. |
| | 616 |
| | The corneal simKs pre-populate |
| | with the ability to override. |
| | 618 |
| | The user enters SIA power and |
| | incision location. SIA could be O |
| | if the user doesn't expect |
| | additional astigmatism if using the |
| | same incision location as the first |
| | procedure. |
| | 620 |
| | The software will calculate the |
| | lens rotation necessary to correct |
| | the astigmatism. |
| 610b | 622 |
| User clicks OK | The software displays the entered |
| | and calculated information on |
| | screen. It does not display the |
| | lens options or the Select Lens |
| | button. |
| | 624 |
| | The software displays the current |
| | steep/flat corneal axis, the |
| | potential induced steep/flat axis |
| | and includes a new line to indicate |
| | the current lens placement axis. |
| | 626 |
| | A button on the screen allows the |
| | user to show or hide the new |
| | planned lens placement. |
| 630 | 632 |
| User click Show New Lens | The new lens placement symbol |
| Placement | appears on-screen with angle |
| | measurements between the |
| | current lens axis and the planned |
| | lens axis. |

If necessary or desired the New Lens Placement can be changed and the lens placement symbol will appear with angle measurements between the current lens axis and the new planned lens axis until an optimum placement is obtained.

### Post-Operative Plan for ICL Implant

The software modules described herein enable a user to design a post-operative plan, if the patient's astigmatism still requires correction after implantation of a toric ICL, as shown in Table 7.

TABLE 7

| Action | Software Response |
|---|---|
| 710 | 712 |
| After choosing a post-op | The software displays a screen per GUI |
| wavefront or corneal | requirements and displays the CT eye |
| topography exam, the user | image. |
| clicks the ToricICL button | 714 |
| or icon | A reference line is placed at the 0 and |
| | 180 axis. The user activates the toric |
| | caliper by clicking outside the circle grid, |
| | similarly to the Toric Planner (the user |
| | does not have to click show caliper). |
| | 716 |
| | The calipers can be moved to the |
| | desired locations. One or two caliper |
| | lines appear that measure the angle |
| | from the closest reference line endpoint |
| | to the caliper. In the case of one caliper, |
| | it will measure the angle from both ends |
| | of the reference line. |
| 720a | 722 |
| User clicks Data Entry button | The software provides a form for |
| | entering lens data, such as sphere and |
| | cyl power and axis, manufacturer and |
| | model (for availability on surgical plan |
| | printout). |
| 720b | 724 |
| User clicks OK in data entry | The entered data appears on screen. |
| 730 | 730 |
| User clicks Show Axial Map | The translucent axial topography map |
| | displays |
| 740 | 742 |
| User clicks Surgeon's View | The map rotates to view from the |
| | superior and indicates that this is the |
| | surgical plan |

After step 740, the surgical plan can be edited as described herein.

One skilled in the art will appreciate readily that the present invention is well adapted to carry out the objects and obtain the ends and advantages mentioned, as well as those objects, ends and advantages inherent herein. The present examples, along with the methods, systems procedures and treatments described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A measurement tool for implantable non-spherical asymmetric optics, comprising:
a viewable rotatable angular caliper superimposable over an image of an eye, said caliper comprising:
a pair of axes through the circle forming the angular caliper and intersecting at a point corresponding to a corneal vertex when superimposed over the eye; and
a plurality of markings around the circumference each corresponding to angular degrees from the axes.

2. The measurement tool of claim 1, wherein the circumference of the caliper superimposes approximately around the limbus of the eye.

3. A method for optimally placing non-spherical asymmetric optics in an eye of a patient, comprising the steps of:
making reference marks at one or more points of interest on an eye;
measuring the corneal topography of the marked eye to map its metrics of a steep axis, a flat axis and an angle of corneal astigmatism;
superimposing the measurement tool of claim 1 over the corneal topographic image of the eye;
determining, via the measurement tool, an optimal angle of an optical zone on the cornea for placement of the non-spherical asymmetric optic; and
positioning the non-spherical asymmetric optic to coincide with the optimal angle of the optical zone.

4. The method of claim 3, further comprising the step of:
measuring residual total astigmatism of the eye after placing the non-spherical asymmetric optic into the eye to determine whether to further minimize or eliminate the residual astigmatism or to leave it to provide depth of focus.

5. The method of claim 4, wherein the residual astigmatism is further minimized or eliminated, the method comprising the steps of:
subtracting corneal astigmatism from the residual total astigmatism to determine the current angle of the implanted non-spherical asymmetric optic;
calculating a rotation of the implanted asymmetric optic required to minimize or eliminate the residual astigmatism;
calculating the angle between the marks on the eye and a new axis of the implanted non-spherical asymmetric optic; and
rotating the implanted non-spherical asymmetric optic the calculated amount to coincide with the new calculated angle.

6. The method of claim 3, wherein the steps of determining the optical zone metrics comprise:
determining the sphero-cylindrical shape that is a best fit to the optical zone of the corneal topography or of a corneal wavefront.

7. The method of claim 3, wherein the step of determining the optimal angle for placement of the non-spherical asymmetric optic comprises:
measuring one or more angles formed by one or more first axes each having a vertex coincident with one of the reference marks and a second axis comprising one of the metrics, said first and second axes each having a vertex coincident with a central vertex in the eye, said non-spherical asymmetric optic position coinciding with the axes.

8. The method of claim 7, wherein the other vertex(ices) of the first axis(es) comprises one of the reference mark(s).

9. The method of claim 7, wherein the second axis is coincident with a steep axis of the corneal topography curvature.

10. The method of claim 7, wherein the central vertex is located in the center of the cornea, the pupil or the entrance pupil or the center of corneal topographic map or is located at a corneal anomaly.

11. The method of claim 3, wherein the corneal topography includes one or both of wavefront or aberrometry measurements or measurements of other optical aberrations.

12. The method of claim 3, wherein the optical aberration is astigmatism.

13. The method of claim 3, wherein the non-spherical asymmetric optics are implantable toric intraocular lenses or implantable toric intraocular contact lenses.

14. A method for correcting astigmatism in vision of a patient having cataract surgery, comprising the steps of:
measuring a corneal topography to pre-determine astigmatism in a cornea of the patient's eye;
determining an angle within an optical zone of interest on the cornea of the eye for an optimal non-spherical astigmatic correction based on metrics determined from the corneal topography;
planning, via the measurement tool of claim 1, surgical placement into the eye of an implantable non-spherical asymmetric optic; and
positioning the implantable non-spherical asymmetric optic to coincide with the optimal angle for the optical zone of interest.

15. The method of claim 14, further comprising the steps of:
measuring residual astigmatism after the implantation;
calculating a new rotation and axis for the implanted non-spherical asymmetric optic required to minimize or to eliminate the residual astigmatism; and
repositioning the implanted non-spherical asymmetric optic thereby further minimizing the post-operative residual astigmatism.

16. The method of claim 14, wherein the step of determining the metrics of the optical zone of interest comprises the step of:
determining the sphero-cylindrical shape that is a best fit to the optical zone.

17. The method of claim 14, wherein the step of determining the angle of the optical zone comprises:
measuring one or more angles formed by one or more first axes each having a vertex coincident with a reference mark placed on the eye and a second axis comprising a metric based on the corneal topography, said first and second axes each having a vertex coincident with a central vertex in the eye, said non-spherical asymmetric optic position coinciding with the axes.

18. The method of claim 17, wherein the central vertex is located in the center of the cornea, the pupil or the entrance pupil or the center of corneal topography or is located at a corneal anomaly.

19. The method of claim 17, wherein the second axis is the steep axis of corneal curvature.

20. The method of claim 14, wherein the non-spherical asymmetric optic is an implantable toric intraocular lens or an implantable toric intraocular contact lens.

21. A computer program product for use in execution in a computer of a method for planning a surgical implantation of non-spherical asymmetric optics into one or both eyes of a patient, said computer having at least a memory and a processor, the computer program product comprising:
a data module configured to input into user-entered fields first values for at least IOL spherical power, surgically induced astigmatism and incision location and to output into calculated fields second values, calculated from the first inputted values, for at least lens data, an axis of placement of the non-spherical asymmetric optics in the one or both eyes and an expected residual astigmatism;
a lens selection module configured to select the non-spherical asymmetric optics based on the calculated values; and
a surgical plan module configured to plan and to display a surgical implantation of the non-spherical asymmetric optics based on the calculated values and the lens selection.

22. The computer program product of claim 21, wherein the inputted first values further comprise one or more of axial length, anterior chamber depth, central corneal thickness, lens thickness, or retinal thickness.

23. The computer program product of claim 21, wherein the outputted calculated values further comprise one or more of pre-operative corneal astigmatism, a cross cylinder result for a corneal plane, cylinder power at the IOL plane, or cylinder power at the corneal plane.

24. The computer program product of claim 21, wherein the data entry module is further configured to edit the inputted first values and recalculate outputted second values based on a post-operative residual astigmatism value.

25. A computer readable medium tangibly storing the instructions for execution in a computer of a method for planning a surgical implantation of non-spherical asymmetric optics into one or both eyes of a patient, said computer having at least a memory and a processor, the method comprising the steps of:
inputting into user-entered fields first values for at least IOL spherical power, surgically induced astigmatism and incision location;
outputting into calculated fields second values, calculated from the first inputted values, for at least lens data, an axis of placement of the non-spherical asymmetric optics in the one or both eyes and an expected residual astigmatism;
selecting the non-spherical asymmetric optics based on the calculated values; and
planning and displaying a surgical implantation of the non-spherical asymmetric optics based on the calculated values and the lens selection.

26. The computer readable medium of claim 25, further comprising the step of:
inputting first values for one or more of axial length, anterior chamber depth, central corneal thickness, lens thickness, or retinal thickness.

27. The computer readable medium of claim 25, further comprising the step of:
outputting calculated values for one or more of pre-operative corneal astigmatism, a cross cylinder result for a corneal plane, cylinder power at the IOL plane, or cylinder power at the corneal plane.

28. The computer readable medium of claim 25, further comprising:
editing the inputted first values and recalculating outputted second values based on a post-operative residual astigmatism value.
